(19) United States
(12) Patent Application Publication
(10) Pub. No.: US 2025/0177419 A1
(43) Pub. Date: Jun. 5, 2025

(54) PHARMACEUTICAL COMPOSITION COMPRISING NICLOSAMIDE OR PHARMACEUTICALLY ACCEPTABLE SALT THEREOF AND PREPARATION METHOD THEREFOR

(71) Applicants: WEBIOTREE CO., LTD, Seoul (KR); CNPHARM CO., LTD., Seoul (KR)

(72) Inventors: Ho Jun KIM, Seoul (KR); Ki Yeok KIM, Seoul (KR); Geun Woo JIN, Seoul (KR)

(73) Assignees: WEBIOTREE CO., LTD, Seoul (KR); CNPHARM CO., LTD., Seoul (KR)

(21) Appl. No.: 18/844,666

(22) PCT Filed: Mar. 8, 2023
(86) PCT No.: PCT/KR2023/003179

§ 371 Date: Sep. 6, 2024

(30) Foreign Application Priority Data
Mar. 8, 2022 (KR) 10-2022-0029601

(51) Int. Cl. A61K 31/609; A61K 9/16; A61K 47/02; A61K 47/10; A61K 47/18; A61K 47/20; A61K 47/32; A61K 47/34; A61K 47/36; A61K 47/38; A61P 31/14

(52) U.S. Cl. CPC A61K 31/609; A61K 9/1611; A61K 9/1635; A61K 9/1652; A61K 47/02; A61K 47/10; A61K 47/183; A61K 47/20; A61K 47/32; A61K 47/34; A61K 47/36; A61K 47/38; A61P 31/14

(57) ABSTRACT

The present invention relates to a pharmaceutical composition containing niclosamide or a pharmaceutically acceptable salt thereof and a method for preparing the same, which can improve the low body absorption rate of niclosamide.

### TECHNICAL FIELD

The present invention relates to a pharmaceutical composition comprising niclosamide or a pharmaceutically acceptable salt thereof and a method for preparing the same, which can improve the low body absorption rate of niclosamide.

### BACKGROUND ART

In the development of oral formulations for poorly soluble drugs, improving dissolution and solubility has become increasingly important to enhance the bioavailability of these drugs. Niclosamide which has demonstrated potent antiviral effects against SARS-CoV-2, the virus responsible for the recent COVID-19 pandemic, is a representative example of a poorly soluble drug with significantly low solubility. Due to the structural nature of the drug, it is difficult for patients to absorb, which imposes limitations on its use. In efforts to overcome this challenge, various studies have been conducted, highlighting that improving the solubility and dissolution of the drug through solubilization is a crucial factor in enhancing drug absorption.

Generally, solid dispersion techniques have been employed for solubilization, using poorly soluble drugs and ionic or non-ionic polymers, surfactants, etc., to convert crystalline drugs into amorphous particles. Accordingly, various polymeric substances have been introduced in studies such as ‘Contribution to the Improvement of an Oral Formulation of Niclosamide, an Antihelmintic Drug Candidate for Repurposing in SARS-CoV-2 and Other Viruses’ (co-authored by Eduardo Jose Barbosa and five others) regarding oral formulations of niclosamide. These disclosed studies suggest that using polymeric substances can improve the solubility of niclosamide. However, the range of enhanced solubility achieved has not yet produced a significant improvement in the actual bioavailability of niclosamide, nor has it demonstrated a substantial antiviral effect in the body.

Prior Art Document: Contribution to the Improvement of an Oral Formulation of Niclosamide, an Antihelmintic Drug Candidate for Repurposing in SARS-CoV-2 and Other Viruses (Eduardo Jose Barbosa and five others)

### DISCLOSURE OF THE INVENTION

### Technical Problem

The present invention relates to an antiviral composition comprising niclosamide or a pharmaceutically acceptable salt thereof, and a method for preparing the same, with the object of providing a composition and preparation method that can enhance the low bioavailability of niclosamide.

In addition, another object of the present invention is to provide a composition for antiviral or anticancer and its preparation method that, by delaying the crystallization of the drug in the body, improves bioavailability, and ensures stable delivery of the drug to the intestines by minimizing the effects of bodily fluids such as gastric acid or bile acids during oral administration.

### Technical Solution

The present invention provides a composition for antiviral or anticancer comprising niclosamide or a pharmaceutically acceptable salt thereof; a polyvinyl pyrrolidone-based compounds; and one or more compounds selected from a cellulose-based compound, a poloxamer-based compound, a polyethylene glycol-based compound, chitosan, poly-gamma-glutamic acid (γ-PGA), and xanthan gum.

In addition, the present invention provides a method for preparing a composition for antiviral or anticancer, comprising the steps of: preparing a first dissolved product by dissolving a polyvinyl pyrrolidone-based compounds in anhydrous ethanol; preparing a second dissolved product by adding and stirring niclosamide or a pharmaceutically acceptable salt thereof into the first dissolved product; preparing a third dissolved product by further mixing one or more compounds selected from a cellulose-based compound, a poloxamer-based compound, a polyethylene glycol-based compound, poly-gamma-glutamic acid (γ-PGA), chitosan, and xanthan gum into the second dissolved product; and forming powders by evaporating the solvent from the third dissolved product.

Furthermore, the present invention provides a composition for antiviral or anticancer comprising niclosamide or a pharmaceutically acceptable salt thereof; and one or more compounds selected from poly-gamma-glutamic acid and an amino acid-based compound.

### Advantageous Effects

The present invention has the effect of enabling a drug with low bioavailability, such as niclosamide or a pharmaceutically acceptable salt thereof, to achieve a meaningful blood concentration and bioavailability sufficient to attain its intended pharmaceutical effects.

In addition, the present invention provides a composition that delays the crystallization of the drug in the body to improve bioavailability and ensures stable delivery of the drug to the intestines by minimizing the impact of bodily fluids such as gastric acid or bile acids during oral administration.

Moreover, the present invention offers a composition that forms a drug-matrix composition using a polymeric substance for poorly soluble drugs like niclosamide, thereby improving the poor dispersion and maintaining blood concentration, which are issues associated with poorly soluble drugs, to provide excellent bioavailability.

Furthermore, the present invention provides a method for preparing a composition that can overcome the challenges associated with the poor solubility of niclosamide or a pharmaceutically acceptable salt thereof.

### BRIEF DESCRIPTION OF THE DRAWINGS

FIG. 1 is a graph showing the blood concentration of the drug in Compositions of Examples 1 to 3.

FIG. 2 is a graph showing the AUC results of Examples 1 and 2.

FIG. 3 is a graph showing the blood concentration of the drug in Compositions of Comparative Examples 1 and 2.

FIG. 4 is a graph showing the blood concentration of the drug in Compositions of Reference Examples 9 to 11 and Examples 2 to 6.

FIG. 5 is a graph showing the AUC results of Reference Examples 9 to 11 and Examples 2 to 6.

FIG. 6 is a graph showing the blood concentration of the drug in Compositions of Examples 7 to 10.

FIG. 7 is a graph showing the AUC results of Examples 7 to 10.

FIG. 8 is a graph showing the blood concentration of the drug in Examples 11 and 12, and Comparative Example 3 (Yomesan).

FIG. 9 is a graph showing the AUC results of the Compositions of Examples 11 and 12, and Comparative Example 3.

FIGS. 10 to 13 are graphs showing the dissolution rates of each composition at different pH levels.

FIG. 14 is a schematic diagram of the antiviral efficacy and cytotoxicity analysis experiments for niclosamide and remdesivir.

FIG. 15 is a graph and table showing that niclosamide or a pharmaceutically acceptable salt thereof inhibited the in vivo proliferation of the Omicron virus 2 to 4 times more effectively compared to the control group (remdesivir). Specifically, the IC₅₀ and CC₅₀ of the compounds were measured in Vero E6 cells and compared to the untreated group. The viral growth reduction rate of the treated group is shown in red, and cell viability is shown in blue.

FIG. 16 provides detailed results of Experimental Example 5.

### MODE FOR CARRYING OUT THE INVENTION

The present invention relates to a composition that can enhance the bioavailability of a drug in vivo, specifically a composition for antiviral or anticancer comprising niclosamide or a pharmaceutically acceptable salt thereof; a polyvinyl pyrrolidone-based compounds; and one or more compounds selected from a cellulose-based compound, a poloxamer-based compound, a polyethylene glycol-based compound, chitosan, and xanthan gum.

The present invention also provides a composition for antiviral or anticancer that can enhance the bioavailability of a drug in vivo, comprising niclosamide or a pharmaceutically acceptable salt thereof; and an amino acid-based compound.

### MODE FOR CARRYING OUT THE INVENTION

In the present invention, “pharmaceutically acceptable salts” refer to salts commonly used in the pharmaceutical industry, such as inorganic ion salts made from calcium, potassium, sodium, magnesium, etc.; inorganic acid salts made from hydrochloric acid, nitric acid, phosphoric acid, hydrobromic acid, iodic acid, perchloric acid, sulfuric acid, etc.; organic acid salts made from acetic acid, trifluoroacetic acid, citric acid, maleic acid, succinic acid, oxalic acid, benzoic acid, tartaric acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, etc.; sulfonic acid salts made from methanesulfonic acid, ethanesulfonic acid, benzenesulfonic acid, p-toluenesulfonic acid, naphthalenesulfonic acid, etc.; amino acid salts made from glycine, arginine, lysine, etc.; and amine salts made from trimethylamine, triethylamine, ammonia, pyridine, picoline, etc. However, the salts mentioned here are not intended to limit the types of salts meant in this invention.

In this invention, it has been confirmed that when combining one or more of cellulose-based compounds, poloxamer-based compounds, polyethylene glycol-based compounds, chitosan, poly-gamma-glutamic acid, and xanthan gum with niclosamide and a polyvinyl pyrrolidone-based compounds, the resulting composition can achieve superior bioavailability compared to the simple combination of niclosamide and the polyvinyl pyrrolidone-based compounds alone.

In particular, polyvinyl pyrrolidone-based compounds are known for their crystallization inhibition properties, and experiments have been conducted on compositions combining these compounds with niclosamide. However, merely combining the two has shown limitations in sufficiently improving the bioavailability of niclosamide. Therefore, to achieve a higher pharmacological effect of poorly soluble drugs, it has been confirmed that, by loading niclosamide or a pharmaceutically acceptable salt thereof into a polymer compound that either inhibits crystallization or possesses swelling properties in combination with the polyvinyl pyrrolidone-based compounds, it is possible to delay the crystallization time of the drug, increase its solubility in the body, and improve the blood concentration of the drug.

Specifically, the polyvinyl pyrrolidone-based compounds can be selected from one or more of the group consisting of polyvinylpyrrolidone K10 (MW 8,000-10,000), polyvinylpyrrolidone K12 (MW 11,000-12,000), polyvinylpyrrolidone K15 (MW 14,000-18,000), polyvinylpyrrolidone K17 (MW 14,000-18,000), polyvinylpyrrolidone K18 (MW 14,000-18,000), polyvinylpyrrolidone K25 (MW 20,000-25,000), polyvinylpyrrolidone K30 (MW 30,000-40,000), polyvinylpyrrolidone K60 (MW 50,000-60,000), and polyvinylpyrrolidone K90 (MW 80,000-90,000). Here, “MW” refers to molecular weight, which means the weight-average molecular weight.

The cellulose-based compounds can be selected from one or more of the group consisting of hydroxypropyl methylcellulose (HPMC), hydroxyethyl cellulose, hydroxypropyl cellulose (HPC), carboxymethylcellulose (CMC), ethylcellulose (EC), methylcellulose (MC), and cellulose acetate (CA). The weight-average molecular weight of the aforementioned cellulose-based compounds can range from 5,000 to 500,000.

The poloxamer-based compounds can be selected from one or more of the group consisting of poloxamer 101, poloxamer 105, poloxamer 105 benzoate, poloxamer 108, poloxamer 122, poloxamer 123, poloxamer 124, poloxamer 181, poloxamer 182, poloxamer 182 dibenzoate, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, with poloxamer 407 being more preferred. The weight-average molecular weight of the aforementioned poloxamer-based compounds can range from 5,000 to 500,000.

The polyethylene glycol-based compounds may be selected from one or more of the group consisting of polyethylene glycol 200, polyethylene glycol 300, polyethylene glycol 400, polyethylene glycol 500, polyethylene glycol 1000, polyethylene glycol 1400, polyethylene glycol 1500, polyethylene glycol 4000, polyethylene glycol 8000, polyethylene glycol 10000, and methoxy polyethylene glycol 550. The weight-average molecular weight of the aforementioned polyethylene glycol-based compounds can range from 5,000 to 500,000.

The present invention also confirmed that when niclosamide or a pharmaceutically acceptable salt thereof is combined with poly-gamma-glutamic acid and one or more amino acids, it can achieve superior bioavailability compared to simply using niclosamide alone.

Specifically, the amino acids can include nonpolar amino acids, polar amino acids, acidic amino acids, and basic amino acids. More specifically, the nonpolar amino acids may include glycine, alanine, valine, leucine, isoleucine, methionine, phenylalanine, tryptophan, and proline; the polar amino acids may include serine, threonine, cysteine, tyrosine, asparagine, and glutamine; the acidic amino acids may include aspartic acid and glutamic acid; and the basic amino acids may include lysine, arginine, and histidine. Among these amino acids, those with a side chain pka value of 3 or higher are more preferred. These include cysteine (side chain pKa 8.33), tyrosine (side chain pKa 10.07), aspartic acid (side chain pKa 3.86), glutamic acid (side chain pKa 4.25), lysine (side chain pKa 10.79), arginine (side chain pKa 12.48), and histidine (side chain pKa 6.04). Using these amino acids can increase the solubility of niclosamide in slightly alkaline solutions.

The composition for antiviral or anticancer of the present invention may further include one or more selected from magnesium oxide (MgO), hydrotalcite, and magnesium hydroxide. Including one or more selected from magnesium oxide, hydrotalcite, and magnesium hydroxide can further enhance the blood solubility of niclosamide, resulting in a significantly improved bioavailability, which is highly desirable.

The composition for antiviral or anticancer of the present invention may include one or more combinations selected from the following: a first combination comprising niclosamide or a pharmaceutically acceptable salt thereof, a polyvinyl pyrrolidone-based compounds, and a cellulose-based compound; a second combination comprising niclosamide, a polyvinyl pyrrolidone-based compounds, and a poloxamer-based compound; a third combination comprising niclosamide, a polyvinyl pyrrolidone-based compounds, and a polyethylene glycol-based compound; a fourth combination comprising niclosamide, a polyvinyl pyrrolidone-based compounds, and chitosan; a fifth combination comprising niclosamide, a polyvinyl pyrrolidone-based compounds, and xanthan gum; a sixth combination comprising niclosamide, a polyvinyl pyrrolidone-based compounds, a cellulose-based compound, and a poloxamer-based compound; a seventh combination comprising niclosamide, a polyvinyl pyrrolidone-based compounds, a cellulose-based compound, and a polyethylene glycol-based compound; an eighth combination comprising niclosamide, a polyvinyl pyrrolidone-based compounds, a cellulose-based compound, and chitosan; a ninth combination comprising niclosamide, a polyvinyl pyrrolidone-based compounds, a cellulose-based compound, and xanthan gum; a tenth combination comprising niclosamide and an amino acid-based compound; an eleventh combination comprising niclosamide, a polyvinyl pyrrolidone-based compounds, and an amino acid-based compound; a twelfth combination comprising niclosamide and poly-gamma-glutamic acid; a thirteenth combination comprising niclosamide, polyvinyl pyrrolidone, and poly-gamma-glutamic acid; a fourteenth combination comprising niclosamide, polyvinyl pyrrolidone, poly-gamma-glutamic acid, and an amino acid-based compound; and a fifteenth combination comprising niclosamide, poly-gamma-glutamic acid, and an amino acid.

Specifically, an example of the first combination may be a composition comprising niclosamide, polyvinyl pyrrolidone, and hydroxypropyl methylcellulose; an example of the second combination may be a composition comprising niclosamide, polyvinyl pyrrolidone, and poloxamer; an example of the third combination may be a composition comprising niclosamide, polyvinyl pyrrolidone, and polyethylene glycol 1400; an example of the fourth combination may be a composition comprising niclosamide, polyvinyl pyrrolidone, and chitosan; an example of the fifth combination may be a composition comprising niclosamide, polyvinyl pyrrolidone, and xanthan gum; an example of the sixth combination may be a composition comprising polyvinyl pyrrolidone-based compounds, hydroxypropyl methylcellulose, and poloxamer; an example of the seventh combination may be a composition comprising niclosamide, polyvinyl pyrrolidone, hydroxypropyl methylcellulose, and polyethylene glycol 1400; an example of the eighth combination may be a composition comprising niclosamide, polyvinyl pyrrolidone, hydroxypropyl methylcellulose, and chitosan; an example of the ninth combination may be a composition comprising niclosamide, polyvinyl pyrrolidone, hydroxypropyl methylcellulose, and xanthan gum; an example of the tenth combination may be a composition comprising niclosamide and arginine; an example of the eleventh combination may be a composition comprising niclosamide, polyvinyl pyrrolidone, and arginine; an example of the twelfth combination may be a composition comprising niclosamide and poly-gamma-glutamic acid; an example of the thirteenth combination may be a composition comprising niclosamide, polyvinyl pyrrolidone, and poly-gamma-glutamic acid; an example of the fourteenth combination may be a composition comprising niclosamide, polyvinyl pyrrolidone, poly-gamma-glutamic acid, and arginine; and an example of the fifteenth combination may be a composition comprising niclosamide, poly-gamma-glutamic acid, and arginine.

The composition for antiviral or anticancer of the present invention may further include one or more selected from magnesium oxide (MgO), hydrotalcite, and magnesium hydroxide in the above combinations 1 to 15. Including one or more of magnesium oxide, hydrotalcite, and magnesium hydroxide can further improve the blood solubility of improved niclosamide, thereby resulting in significantly bioavailability, which is desirable.

The composition for antiviral or anticancer of the present invention may further include one or more substances selected from the group consisting of crystallization inhibitors, swellable polymers, enteric coatings, foam generators, and swellable excipients.

Specifically, in the present invention, a crystallization inhibitor refers to a polymer that can delay the return of a compound, such as niclosamide, from an amorphous or non-crystalline state to a crystalline form when dissolved in a solvent. By loading the drug onto the crystallization inhibitor and thereby delaying the crystallization time of the drug, the solubility of the drug in the body can be increased, which can, in turn, increase the blood concentration of the drug. Specifically, the crystallization inhibitor may be selected from polyoxyethylene sorbitan fatty acid esters, lecithin-based compounds, fatty acid-based compounds, glycerol fatty acid esters, sorbitan fatty acid esters, oils, sodium dodecyl sulfate, sodium stearyl fumarate, stearic acid, lauric acid, and carrageenan.

In particular, commercially available Tween surfactants are representative examples of polyoxyethylene sorbitan fatty acid esters, which take the form of ester bonds between fatty acids and ethylene oxide. More specifically, they may include polyoxyethylene sorbitan monolaurate (Tween 20), polyoxyethylene sorbitan monopalmitate (Tween polyethylene glycol sorbitan monostearate (Tween 60), Tween 65, polyoxyethylene sorbitan monooleate (Tween 80), and polyoxyethylene sorbitan trioleate (Tween 85).

In addition, the lecithin-based compounds may include lecithin and its derivatives, such as phospholipids, phosphatidylcholine, mixed phospholipids, sodium cholate, hydroxylated phospholipids, and hydroxylated lecithin.

Furthermore, the fatty acid-based compounds may include butyric acid, caproic acid, caprylic acid, capric acid, stearic acid, lauric acid, oleic acid, myristoleic acid, palmitoleic acid, oleic acid, linoleic acid, alpha-linolenic acid, gamma-linolenic acid, gadoleic acid, eicosadienoic acid, eicosapentaenoic acid, arachidonic acid, erucic acid, docosadienoic acid, docosatrienoic acid, docosapentaenoic acid, docosahexaenoic acid, adrenic acid, and nervonic acid.

In addition, the glycerol fatty acid esters may include polyglycerol fatty acid esters, polyglycerol polyricinoleate, polyoxyethylene glycerol triricinoleate, and Cremophor EL.

In addition, the sorbitan fatty acid esters may include sorbitan monolaurate (Span 20) and sorbitan monooleate (Span 80).

In addition, the oils may include soybean oil, MCT oil (Medium-Chain Triglyceride), and castor oil.

Including the aforementioned crystallization inhibitors is desirable as they can improve the solubility and dispersion of poorly soluble drugs.

In the present invention, the swellable polymers are substances that can help control the drug release rate during formulation. These may include calcium carboxymethylcellulose, sodium carboxymethylcellulose, methylcellulose, ethyl cellulose, polyethylene oxide, locust bean gum, guar gum, xanthan gum, acacia gum, tragacanth gum, alginic acid, sodium alginate, calcium alginate, ammonium alginate, agar, gelatin, polymethacrylate, carbomer, polycarbophil, polyvinyl acetate, polyvinylpyrrolidone-polyvinyl acrylate copolymer, polyvinyl alcohol-polyethylene glycol copolymer, polyvinylpyrrolidone-polyvinyl acetate copolymer, bentonite, hectorite, carrageenan, carob (Ceratonia), ceto stearyl alcohol, hydroxypropyl starch, magnesium aluminum silicate, polydextrose, poly(methyl vinyl ether/maleic anhydride), propylene glycol alginate, and saponins.

In the present invention, the enteric coating refers to a substance that prevents the drug from being recrystallized by gastric fluid, which can hinder absorption in the stomach, thus increasing the drug's absorption rate. Using an enteric coating allows for the selection of the specific region in the intestines where the drug is released.

In particular, for niclosamide, the bioavailability varies over time depending on the pH of different intestinal regions, making the use of enteric coatings desirable to select more optimized formulation. Specifically, the enteric coating may be selected from hydroxypropyl methylcellulose phthalate, zein, shellac, and Eudragit.

In the present invention, the foam generators may be selected from the group consisting of sodium carbonate, sodium bicarbonate, potassium carbonate, potassium bicarbonate, and citric acid, but are not limited to these.

In addition, the swellable excipients may include carboxymethylcellulose, natural cellulose, pectin, hyaluronic acid, polyacrylate, polyethylene oxide, polypropylene oxide, monosaccharides, methacrylic acid-ethyl acrylate copolymers, shellac, carbopols (carbomer, carboxyvinyl polymer), polyvinyl alcohol, hydroxypropyl methylcellulose phthalate-based compounds, cellulose acetate phthalate, cellulose acetate succinate, hydroxypropyl methylcellulose acetate succinate, hydroxypropyl methyl acetate succinate, carboxymethylcellulose, carboxymethyl ethylcellulose, cellulose acetate phthalate-based compounds, hydroxypropyl cellulose-based compounds, ethylcellulose-based compounds, methylcellulose-based compounds, polyvinyl acetate phthalate, silica, calcium silicate, lactose, starch, mannitol, kaolin inorganic salts, powdered sugars, powdered cellulose derivatives, and microcrystalline cellulose. Commonly used swellable excipients are not limited and can be used.

The composition of the present invention, when formulated into dosage forms such as film coating, semi-permeable coating, water-insoluble coating, tablets, bilayer tablets, or gastric retention tablets using the above-mentioned coatings, was found to result in significantly higher blood drug concentrations. Therefore, forming such coatings can help improve the drug's solubility and other effects.

In the present invention, the niclosamide or pharmaceutically acceptable salt thereof and the polyvinyl pyrrolidone-based compounds may satisfy a weight ratio range of 1:2 to 1:12. If this range is not satisfied, the solubility of the poorly soluble drug niclosamide may not sufficiently increase. In addition, if one or more of the cellulose-based compounds, poloxamer-based compounds, polyethylene glycol-based compounds, and polyvinyl pyrrolidone-based compounds exceed a weight ratio of 12, the drug content may decrease, reducing the commercialization potential and causing inefficiencies in drug release, absorption, and digestion behavior within the body.

The composition for antiviral or anticancer of the present invention may further include a pharmaceutically acceptable carrier and can be formulated for oral or non-oral administration for use in humans or animals. When formulating the composition for antiviral or anticancer of the present invention, diluents or excipients such as fillers, extenders, binders, wetting agents, disintegrants, and surfactants may be used in addition to the listed components. Solid preparations for oral administration include tablets, pills, powders, granules, and capsules, and these solid preparations can be formulated by mixing the composition for antiviral or anticancer containing the compound of the present invention with at least one excipient, such as starch, calcium carbonate, sucrose, lactose, gelatin, etc. In addition to simple excipients, lubricants such as magnesium stearate and talc can also be used. Liquid preparations for oral administration include suspensions, solutions, emulsions, and syrups, which can contain various excipients, such as wetting agents, sweeteners, flavoring agents, and preservatives, in addition to the commonly used simple diluents like water and liquid paraffin. Preparations for non-oral administration include sterile aqueous solutions, non-aqueous solvents, suspensions, emulsions, freeze-dried preparations, and suppositories. Non-aqueous solvents and suspensions may include vegetable oils such as propylene glycol, polyethylene glycol, olive oil, and injectable esters such as ethyl oleate. Suppository bases may include witepsol, macrogol, Tween 61, cacao butter, lauric butter, glycerogelatin, and others.

The composition for antiviral or anticancer of the present invention can be administered orally or non-orally, depending on the desired method. For non-oral administration, it is preferable to choose a method such as topical application, intraperitoneal injection, rectal injection, subcutaneous injection, intravenous injection, intramuscular injection, or intrathoracic injection. However, oral administration is most preferable for the composition for antiviral or anticancer of the present invention.

In addition, the composition for antiviral or anticancer according to the present invention can be administered by inhalation. This allows the drug to be delivered directly to the lungs while avoiding toxicity and providing a longer duration of action even at lower doses. Inhalation administration can be achieved using a pharmaceutical preparation containing inhalable particles or droplets that can be inhaled through the airways or nasal passages. While not limited to these devices, examples include a dry powder inhaler (DPI) or a pressurized metered-dose inhaler (pMDI). The drug particles may be lightly compressed within a frangible matrix contained within the delivery device (dry powder inhaler). Upon activation, the delivery device abrades a portion of the drug particles from the matrix and disperses them into the inhalation breath, delivering the drug particles to the airways. Alternatively, the drug particles may be free-flowing powder contained within a reservoir in the delivery device (dry powder inhaler). The reservoir may be an integral chamber within the device or a capsule, blister, or similar reservoir inserted into the device before activation. Upon activation, the device disperses a portion of the drug particles from the reservoir into the inhalation breath, delivering the drug particles to the airways.

The composition of the present invention is administered in a pharmaceutically effective amount. As used in the present invention, the term “pharmaceutically effective amount” refers to an amount sufficient to treat a disease with an acceptable benefit/risk ratio applicable to medical treatment. The effective dosage level can be determined based on factors such as the patient's weight, gender, age, health condition, severity of the disease, activity of the drug, sensitivity to the drug, time of administration, route of administration, and excretion rate, as well as the duration of treatment, concomitant drugs, and other factors well known in the medical field. The composition for antiviral or anticancer of the present invention may be administered as a single treatment or in combination with other therapies, either sequentially or simultaneously with conventional therapies. It may be administered as a single or multiple doses. It is important to administer an amount that achieves maximum efficacy with minimal side effects, considering all of the above factors, and this amount can be easily determined by one skilled in the art.

For example, the composition for antiviral or anticancer according to the present invention can be administered at a dosage of 0.0001 to 500 mg/kg, preferably 0.001 to 500 mg/kg, and the administration may be one to five times a day. In addition, the administration may be once every two days to once every ten days, depending on the purpose.

The composition for antiviral or anticancer of the present invention may also possess anti-inflammatory and/or anti-aging functions in addition to its antiviral or anticancer functions.

In the present invention, “anti-inflammatory” refers to the ability to alleviate inflammatory responses caused by infectious, traumatic, endogenous, inflammatory, degenerative, or autoimmune conditions. This includes diseases such as ulcerative colitis, inflammatory bowel disease, Crohn's disease, and viral enteritis.

In the present invention, “antiviral” refers to effects, which include inhibiting viral having antiviral replication and having antimicrobial functions against viruses such as malaria-causing infections, Epstein-Barr Virus (EBV), Hepatitis B Virus, Hepatitis C Virus, HIV, HTLV-1, Varicella-Zoster Virus (VZV), Human Papillomavirus (HPV), SARS-CoV and/or SARS-CoV2 coronaviruses, rhinoviruses causing colds or respiratory illnesses, adenoviruses, respiratory syncytial virus (RSV), parainfluenza virus, and other retroviruses.

In addition, “anticancer” in the present invention refers to acting primarily on DNA to block DNA replication, transcription, and translation processes, or to inhibit nucleic acid precursor synthesis in metabolic pathways, thereby exhibiting anticancer activity. Specifically, this includes cancers such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma, as well as carcinomas originating in the breast, prostate, kidney, bladder, or colon tissues; lipomatous tumors such as lipoma, fibrolipoma, lipoblastoma, lipomatosis, hibernoma, hemangioma, and/or liposarcoma, and having therapeutic effects on these neoplastic diseases.

In the present invention, “anti-aging” refers to the ability to suppress aging or frailty, where the function of cells and/or necessary gene expression declines, or the body's physiological homeostasis rapidly deteriorates, leading to vulnerability to even minor internal and external stress, resulting in easy onset of diseases or decline in organ function. Diseases associated with aging and/or frailty include conditions caused by brain inflammation, such as dementia and neurodegenerative diseases, and “anti-aging” refers to the prevention, treatment, or suppression of conditions such as dementia and neurodegenerative diseases.

The composition of the present invention can be administered at a dosage of 0.1 mg/kg to 500 mg/kg, one to eight times per day, and the administration can be adjusted to once to three times every two days, three days, four days, or five days, depending on the progression. The composition of the present invention can be administered orally (oral administration), by injection into a vein (intravenous, IV), muscle (intramuscular, IM), the space around the spinal cord (epidural), or just under the skin (subcutaneous, SC). It can also be administered sublingually (under the tongue) or buccally (between the gum and cheek), rectally (rectal administration), vaginally (vaginal administration), into the eye (ocular route), or into the ear (aural route). In addition, it can be administered by spraying into the nose for absorption through the nasal mucosa (nasal administration), by inhalation, by respiratory methods through the mouth and nose to deliver the drug to the lungs, topically to the skin for local or systemic effects, or through the skin using a patch for systemic effects (transdermal administration).

The present invention provides a method for producing a composition for antiviral or anticancer that includes: dissolving a polyvinyl pyrrolidone-based compounds in an organic solvent to prepare a first dissolved product; adding and stirring niclosamide or a pharmaceutically acceptable salt thereof to the first dissolved product to prepare a second dissolved product; mixing one or more compounds selected from cellulose-based compounds, poloxamer-based compounds, polyethylene glycol-based compounds, poly-gamma-glutamic acid, chitosan, and xanthan gum into the second dissolved product to prepare a third dissolved product; and evaporating the solvent from the third dissolved product to produce a powder. In the above, the designation of the first, second, and third dissolved products is arbitrary, and the order of preparing the dissolved products can be changed or substituted, which is obvious to those skilled in the art and falls within the scope of the present invention.

In addition, the present invention provides a method for producing a composition for antiviral or anticancer that includes: dissolving one or more compounds selected from polyvinyl pyrrolidone-based compounds, cellulose-based compounds, poloxamer-based compounds, polyethylene glycol-based compounds, poly-gamma-glutamic acid, chitosan, and xanthan gum in an organic solvent to prepare a first dissolved product; and adding and stirring niclosamide or a pharmaceutically acceptable salt thereof into the first dissolved product to prepare a second dissolved product. The designation of the first and second dissolved products is arbitrary, and the order of preparing the dissolved products can be changed or substituted, which is obvious to those skilled in the art and falls within the scope of the present invention.

The manufacturing method of the present invention may further include a step of drying one or more of the second dissolved product and the third dissolved product to evaporate the solvent and obtain a powder. The powder produced may be mixed in powder form with one or more compounds selected from cellulose-based compounds, poloxamer-based compounds, polyethylene glycol-based compounds, poly-gamma-glutamic acid, chitosan, xanthan gum, magnesium oxide, hydrotalcite, and magnesium hydroxide, followed by milling and/or grinding.

The organic solvent may be selected from ethanol, methanol, propanol, butanol, and acetonitrile. However, in terms of improving reactivity, anhydrous organic solvents may be more preferable, with anhydrous ethanol being the most preferred.

In the manufacturing method of the present invention, water may optionally be added to the organic solvent, or water alone may be used.

The method for producing the composition for antiviral or anticancer of the present invention may further include the step of dissolving and mixing one or more compounds selected from magnesium oxide (MgO), hydrotalcite, and magnesium hydroxide in the second dissolved product and/or third dissolved product. Mixing magnesium oxide during the dissolution process can significantly improve the solubility of poorly soluble drugs, leading to the desired improvement in bioavailability.

In addition, the method for producing the composition for antiviral or anticancer of the present invention may include evaporating the third dissolved product to produce a powder and/or mechanically mixing the powder with one or more compounds selected from magnesium oxide (MgO), hydrotalcite, and magnesium hydroxide. In the present invention, mechanical mixing refers to a method of physically mixing solid substances, which can be done using methods such as mixing, milling, or grinding.

The method for producing the composition for antiviral or anticancer of the present invention may further include stirring the third dissolved product with magnesium oxide and then drying to produce a powder. In the present invention, drying refers to any method of evaporating the solvent, with spray drying being the most preferred method.

The method for producing the composition for antiviral or anticancer of the present invention is characterized by using a solvent evaporation method instead of a hot-melt method, which is generally used in the preparation of solid dispersions. The hot-melt method has disadvantages, such as temperature control during the process and particle attrition in the equipment, which are not suitable for formulating poorly soluble drugs due to the use of expensive equipment like screw extruders. Therefore, the present invention improves manufacturing ease by using a solvent evaporation method, utilizing commonly used ethanol solvents with a rotary evaporator or spray dryer.

The weight ratio of the polyvinyl pyrrolidone-based compounds to niclosamide or a pharmaceutically acceptable salt thereof in the composition for antiviral or anticancer of the present invention may be 2:1 to 12:1.

The composition for antiviral or anticancer of the present invention may include 0.1 to 50% by weight of niclosamide or a pharmaceutically acceptable salt thereof, 0.2 to 95% by weight of polyvinylpyrrolidone (PVP), and 0.5 to 50% by weight of one or more compounds selected from cellulose-based compounds, poloxamer-based compounds, polyethylene glycol-based compounds, chitosan, and xanthan gum, based on the total weight of the composition being 100% by weight. The composition may also further include 0.1 to 70% by weight of one or more selected from magnesium oxide (MgO), hydrotalcite, and magnesium hydroxide, and 0.001 to 30% by weight of one or more substances selected from the group consisting of crystallization inhibitors, swellable polymers, enteric coatings, foam generators, and swellable excipients.

### Examples 1 to 20: Preparation of the Composition of

the Present Invention

### Example 1

In a round-bottom flask, 4 g of PVP is dissolved in 50 mL of anhydrous ethanol, and 1 g of niclosamide is added to the solution and stirred for 1 hour. Then, 1 g of HPMC (6 mPas) is added and stirred for 1 hour. The solvent is completely removed using a rotary evaporator to obtain yellowish powder.

### Example 2

In a round-bottom flask, 4 g of PVP is dissolved in 50 mL of anhydrous ethanol, and 1 g of niclosamide is added to the solution and stirred for 1 hour. Then, 1 g of poloxamer and 1 g of HPMC (6 mPas) are added and stirred for 1 hour. The solvent is completely removed using a rotary evaporator to obtain a yellowish powder.

### Example 3

In a round-bottom flask, 4 g of PVP is dissolved in 50 mL of anhydrous ethanol, and 1 g of niclosamide is added to the solution and stirred for 1 hour. Then, 0.18 g of chitosan and 2 g of MgO are added and stirred for 1 hour. The solvent is completely removed using a rotary evaporator to obtain a yellowish powder.

### Example 4

In a round-bottom flask, 4 g of PVP is dissolved in 50 mL of anhydrous ethanol, and 1 g of niclosamide is added to the solution and stirred for 1 hour. Then, 1 g of HPMC (6 mPas), 1 g of poloxamer, and 2 g of MgO are added and stirred for 1 hour. The solvent is completely removed using a rotary evaporator to obtain a yellowish powder.

### Example 5

In a round-bottom flask, 8 g of PVP is dissolved in 50 mL of anhydrous ethanol, and 1 g of niclosamide is added to the solution and stirred for 1 hour. Then, 1 g of HPMC (6 mPas), 1 g of poloxamer, and 2 g of MgO are added and stirred for 1 hour. The solvent is completely removed using a rotary evaporator to obtain a yellowish powder.

### Example 6

In a round-bottom flask, 8 g of PVP is dissolved in 50 mL of anhydrous ethanol, and 1 g of niclosamide is added to the solution and stirred for 1 hour. Then, 1 g of PEG 1400 is added and stirred for 1 hour. The solvent is completely removed using a rotary evaporator to obtain a yellowish powder.

### Example 7

In a round-bottom flask, 4 g of PVP is dissolved in 20 mL of anhydrous ethanol, and 1 g of niclosamide is added to the solution and stirred for 1 hour. Then, 2 g of MgO and 0.53 g of arginine are added and stirred for 1 hour. The solvent is completely removed using a rotary evaporator to obtain a yellowish powder.

### Example 8

In a round-bottom flask, 4 g of PVP is dissolved in 20 mL of anhydrous ethanol, and 1 g of niclosamide is added to the solution and stirred for 1 hour. Then, 2 g of MgO, 0.2 g of poly gamma glutamic acid (PGA), and 0.53 g of arginine are added and stirred for 1 hour. The solvent is completely removed using a rotary evaporator to obtain a yellowish powder.

### Example 9

In a round-bottom flask, 4 g of PVP is dissolved in 20 mL of anhydrous ethanol, and 1 g of niclosamide is added to the solution and stirred for 1 hour. Then, 2 g of MgO, 0.2 g of PGA, 0.5 g of sodium dodecyl sulfate (SDS), and 0.53 g of arginine are added and stirred for 1 hour. The solvent is completely removed using a rotary evaporator to obtain a yellowish powder.

### Example 10

In a round-bottom flask, 1 g of niclosamide is dissolved in 20 mL of anhydrous ethanol. Then, 2 g of MgO, 0.2 g of PGA, 0.5 g of SDS, and 0.53 g of arginine are added and stirred for 1 hour. The solvent is completely removed using a rotary evaporator to obtain a yellowish powder.

### Example 11

In a round-bottom flask, 1 g of niclosamide is dissolved in 50 mL of anhydrous ethanol. Then, 1 g of MgO and 0.3 g of arginine are added and stirred for 1 hour. The solvent is completely removed using a rotary evaporator to obtain a yellowish powder (granules). The granules are coated using a fluidized bed coater with an enteric coating composition of HP-50 at a coating ratio of 10%.

### Example 12

In a round-bottom flask, 1 g of niclosamide is dissolved in 50 mL of anhydrous ethanol. Then, 1 g of MgO is added and stirred for 1 hour. The solvent is completely removed using a rotary evaporator to obtain a yellowish powder (granules). The granules are coated with an enteric coating composition of Eudragit L-100 at a coating ratio of 5%, and the solvent is completely removed using a rotary evaporator to obtain a yellowish powder.

### Example 13

In a round-bottom flask, 1 g of PVP is dissolved in 50 mL of anhydrous ethanol, and 1 g of niclosamide is added to the solution and stirred for 1 hour. Then, 1 g of MgO is added and stirred for 1 hour. The solvent is completely removed using a rotary evaporator to obtain a yellowish powder.

### Example 14

In a round-bottom flask, 2 g of PVP is dissolved in 50 mL of anhydrous ethanol, and 1 g of niclosamide is added to the solution and stirred for 1 hour. Then, 1 g of MgO is added and stirred for 1 hour. The solvent is completely removed using a rotary evaporator to obtain a yellowish powder.

### Example 15

In a round-bottom flask, 4 g of PVP is dissolved in 50 mL of anhydrous ethanol, and 1 g of niclosamide is added to the solution and stirred for 1 hour. Then, 1 g of MgO is added and stirred for 1 hour. The solvent is completely removed using a rotary evaporator to obtain a yellowish powder.

### Example 16

In a round-bottom flask, 8 g of PVP is dissolved in 50 mL of anhydrous ethanol, and 1 g of niclosamide is added to the solution and stirred for 1 hour. Then, 1 g of MgO is added and stirred for 1 hour. The solvent is completely removed using a rotary evaporator to obtain a yellowish powder.

### Example 17

In a round-bottom flask, 1 g of PVP is dissolved in 50 mL of anhydrous ethanol, and 1 g of niclosamide is added to the solution and stirred for 1 hour. Then, 1 g of poloxamer is added and stirred for 1 hour. After adding 1 g of MgO and stirring for 1 hour, the solvent is completely removed using a rotary evaporator to obtain a yellowish powder.

### Example 18

In a round-bottom flask, 2 g of PVP is dissolved in 50 mL of anhydrous ethanol, and 1 g of niclosamide is added to the solution and stirred for 1 hour. Then, 1 g of poloxamer is added and stirred for 1 hour. After adding 1 g of MgO and stirring for 1 hour, the solvent is completely removed using a rotary evaporator to obtain a yellowish powder.

### Example 19

In a round-bottom flask, 4 g of PVP is dissolved in 50 mL of anhydrous ethanol, and 1 g of niclosamide is added to the solution and stirred for 1 hour. Then, 1 g of poloxamer is added and stirred for 1 hour. After adding 1 g of MgO and stirring for 1 hour, the solvent is completely removed using a rotary evaporator to obtain a yellowish powder.

### Example 20

In a round-bottom flask, 8 g of PVP is dissolved in 50 mL of anhydrous ethanol, and 1 g of niclosamide is added to the solution and stirred for 1 hour. Then, 1 g of poloxamer is added and stirred for 1 hour. After adding 1 g of MgO and stirring for 1 hour, the solvent is completely removed using a rotary evaporator to obtain a yellowish powder.

### Reference Example 1

In a round-bottom flask, 1 g of PVP is dissolved in 50 mL of anhydrous ethanol, and 1 g of niclosamide is added and stirred for about 1 hour. The solvent is completely removed using a rotary evaporator to obtain a yellowish powder.

### Reference Example 2

In a round-bottom flask, 2 g of PVP is dissolved in 50 mL of anhydrous ethanol, and 1 g of niclosamide is added and stirred for about 1 hour. The solvent is completely removed using a rotary evaporator to obtain a yellowish powder.

### Reference Example 3

In a round-bottom flask, 4 g of PVP is dissolved in 50 mL of anhydrous ethanol, and 1 g of niclosamide is added and stirred for about 1 hour. The solvent is completely removed using a rotary evaporator to obtain a yellowish powder.

### Reference Example 4

In a round-bottom flask, 8 g of PVP is dissolved in 50 mL of anhydrous ethanol, and 1 g of niclosamide is added and stirred for about 1 hour. The solvent is completely removed using a rotary evaporator to obtain a yellowish powder.

### Reference Example 5

In a round-bottom flask, 1 g of PVP is dissolved in 50 mL of anhydrous ethanol, and 1 g of niclosamide is added and stirred for about 1 hour. Then, 1 g of poloxamer is added and stirred for 1 hour, and the solvent is completely removed using a rotary evaporator to obtain a yellowish powder.

### Reference Example 6

In a round-bottom flask, 2 g of PVP is dissolved in 50 mL of anhydrous ethanol, and 1 g of niclosamide is added and stirred for about 1 hour. Then, 1 g of poloxamer is added and stirred for 1 hour, and the solvent is completely removed using a rotary evaporator to obtain a yellowish powder.

### Reference Example 7

In a round-bottom flask, 4 g of PVP is dissolved in 50 mL of anhydrous ethanol, and 1 g of niclosamide is added and stirred for about 1 hour. Then, 1 g of poloxamer is added and stirred for 1 hour, and the solvent is completely removed using a rotary evaporator to obtain a yellowish powder.

### Reference Example 8

In a round-bottom flask, 8 g of PVP is dissolved in 50 mL of anhydrous ethanol, and 1 g of niclosamide is added and stirred for about 1 hour. Then, 1 g of poloxamer is added and stirred for 1 hour, and the solvent is completely removed using a rotary evaporator to obtain a yellowish powder.

### Reference Example 9

In a round-bottom flask, 4 g of PVP is dissolved in 50 mL of anhydrous ethanol, and 1 g of niclosamide is added to the solution and stirred for 1 hour. Then, 1 g of HPMC (6 mPas) is added and stirred for 1 hour. The solvent is completely removed using a rotary evaporator to obtain a yellowish powder.

### Reference Example 10

In a round-bottom flask, 4 g of PVP is dissolved in 50 mL of anhydrous ethanol, and 1 g of niclosamide is added to the solution and stirred for 1 hour. Then, 1 g of HPMC (6 mPas) and 1 g of poloxamer are added and stirred for 1 hour. The solvent is completely removed using a rotary evaporator to obtain a yellowish powder.

### Reference Example 11

In a round-bottom flask, 8 g of PVP is dissolved in 50 mL of anhydrous ethanol, and 1 g of niclosamide is added to the solution and stirred for 1 hour. Then, 1 g of sodium dodecyl sulfate is added and stirred for 1 hour. The solvent is completely removed using a rotary evaporator to obtain a yellowish powder.

### Comparative Example 1

In a round-bottom flask, 1 g of Poloxamer 407 is dissolved in 15 mL of anhydrous ethanol, and 1 g of niclosamide and 0.5 g of HPMC (6 mPas) are added to the solution and stirred for 1 hour. The solvent is completely removed using a rotary evaporator to obtain a yellowish powder.

### Comparative Example 2

In a round-bottom flask, 1 g of Poloxamer 407 is dissolved in 15 mL of anhydrous ethanol, and 1 g of niclosamide and 1 g of HPMC (6 mPas) are added to the solution and stirred for 1 hour. The solvent is completely removed using a rotary evaporator to obtain a yellowish powder.

### Experimental Example 1: In Vivo Pharmacokinetic Analysis of the Compositions of the Examples and Comparative Examples (Hamster or Rat)

The in vivo pharmacokinetic analysis was performed using the compositions of Examples 1 to 3 and Comparative Examples 1 and 2. The compositions of Examples 1 to 3 were administered orally to hamsters as a single dose, while Comparative Examples 1 and 2 were administered orally to rats as a single dose. After administration, plasma drug concentration information was obtained.

In addition, the compositions of Examples 1 to 3 were administered at a dosage of 50 mg/kg for the experiment, while Comparative Examples 1 and 2 were also administered at a dosage of 50 mg/kg.

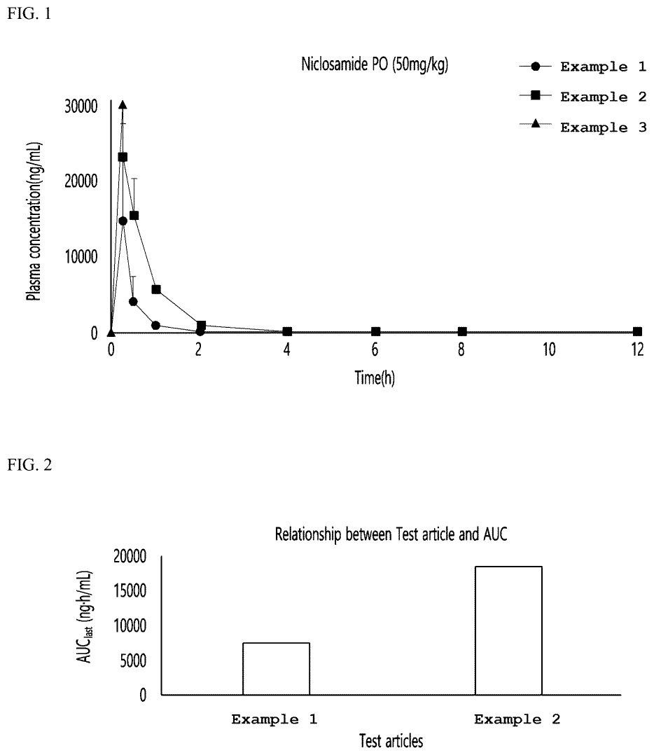

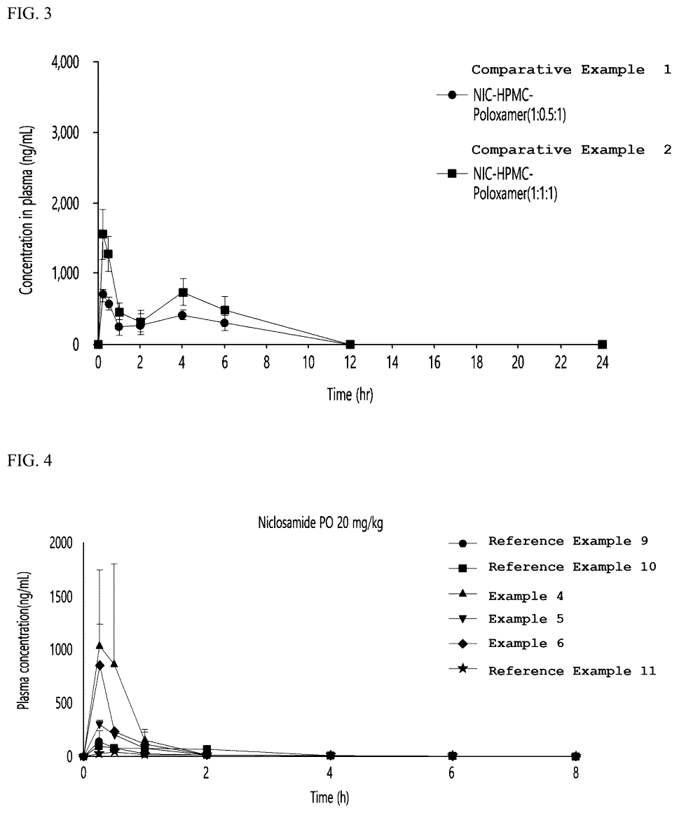

The administration results of Examples 1 to 3 are shown in FIG. 1. The graph in FIG. 1 shows the plasma concentration of NIC over time in hamsters. The specific pharmacokinetic parameters are listed in Table 2, and the relationship between the tested compositions and AUC is shown in FIG. 2. The experimental results revealed that excipients such as PVP, HPMC, and poloxamer contribute to the improvement of bioavailability of niclosamide when formulated, and it was confirmed that the combination of PVP, HPMC, and poloxamer is more effective in enhancing the bioavailability of niclosamide than the combination of PVP and HPMC alone. In addition, the administration results of Comparative Examples 1 and 2 are shown in FIG. 3. The graph in FIG. 3 shows the plasma concentration of NIC over time in rats. This experiment also confirmed that the excipients HPMC and poloxamer can influence not only the improvement of bioavailability of niclosamide but also the extension of its plasma concentration duration.

TABLE 1

| | Niclosamide PO | | |
|---|---|---|---|
| Time | 50 mg/kg | | |
| h | Example 1 | Example 2 | Example 3 |
| 0 | ND | ND | ND |
| 0.25 | 14838.173 | 23429.772 | 30070.216 |
| 0.5 | 4234.492 | 15561.072 | — ( [symbol] ) |
| 1 | 923.414 | 5775.295 | — ( [symbol] ) |
| 2 | 155.958 | 1079.444 | — ( [symbol] ) |
| 4 | 109.866 | 350.397 | — ( [symbol] ) |
| 6 | 163.998 | 194.309 | — ( [symbol] ) |
| 8 | 188.397 | 444.770 | — ( [symbol] ) |
| 12 | 170.154 | 96.878 | — ( [symbol] ) |

* Plasma concentration: ng/mL
* ND: not detected

TABLE 2

Niclosamide PO

| PK Parameters | Example 1 | Example 2 |
|---|---|---|
| AUC (last) | 7497.09 | 18591.20 |
| Cmax | 14838.17 | 21010.39 |
| Tmax | 0.25 | 0.25 |
| t½ | NC | 1.89 |

* AUC: ng · h/mL, Cmax: ng/mL, Tmax & t½: h
** NC: Not calculated

### Experimental Example 2: In Vivo Pharmacokinetic Analysis of the Compositions of the Examples, Reference Examples, and Comparative Examples (Beagle Dogs)

The in vivo pharmacokinetic analysis was performed using the compositions of Examples 4 to 10 and Reference Examples 9 to 11. The compositions of Examples 4 to 10 and Reference Examples 9 to 11 were administered orally as a single dose to beagle dogs, and plasma drug concentration information was obtained.

In addition, the compositions of Examples 4 to 10 and Reference Examples 9 to 11 were each administered at a dosage of 20 mg/kg once daily.

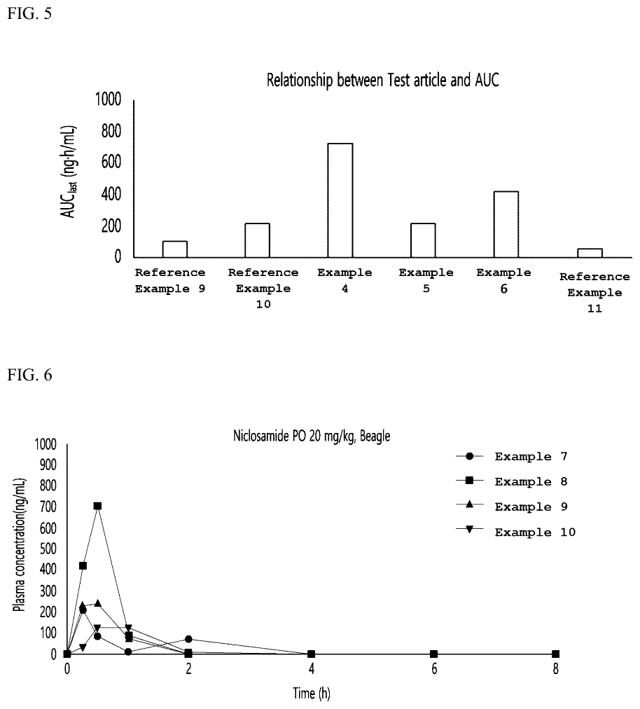

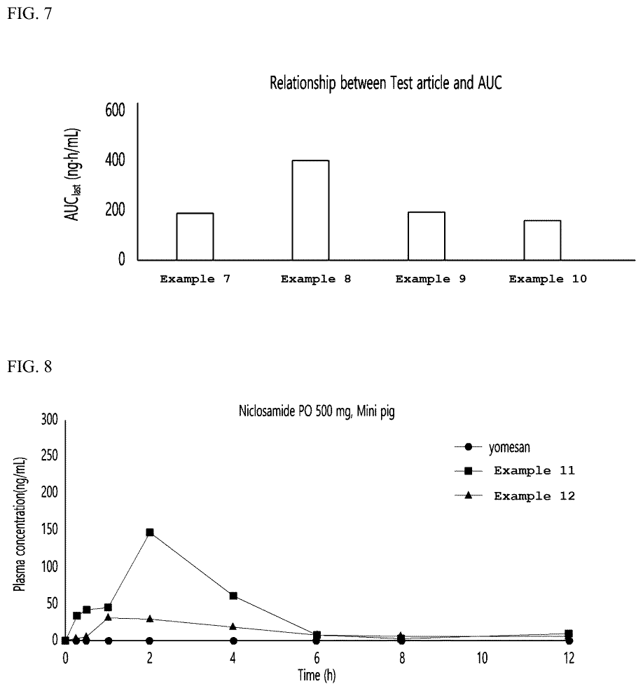

The administration results of Examples 4 to 10 and Reference Examples 9 to 11 are shown in FIGS. 4 to 7. The graphs in FIGS. 4 and 6 show the plasma concentration of NIC over time in beagle dogs. The specific pharmacokinetic parameters are listed in Tables 3 to 6. The relationship between the tested compositions and AUC is shown in FIGS. 5 and 7.

TABLE 3

| | Niclosamide PO 20 mg/kg | | | | | |
|---|---|---|---|---|---|---|
| | Refer- | Refer- | | | | Refer- |
| | ence | ence | | | | ence |
| | Exam- | Exam- | Exam- | Exam- | Exam- | Exam- |
| Time | ple | ple | ple | ple | ple | ple |
| h | 9 | 10 | 4 | 5 | 6 | 11 |
| 0 | ND | ND | ND | ND | ND | ND |
| 0.25 | 135.347 | 90.469 | 1030.266 | 298.21 | 847.999 | 23.757 |
| 0.5 | 80.685 | 72.908 | 855.957 | 201.384 | 233.772 | 37.552 |
| 1 | 23.124 | 85.63 | 153.111 | 75.591 | 114.28 | 13.461 |
| 2 | 11.338 | 64.014 | 16.899 | 7.102 | 16.69 | 10.28 |
| 4 | 2.108 | 1.977 | 1.267 | 0.818 | 1.312 | 4.744 |
| 6 | 1.341 | 0.731 | 0.771 | 0.369 | 3.074 | 0.816 |
| 8 | 0.569 | 0.436 | 0.466 | 0.293 | 1.899 | 0.654 |

*160* Plasma concentration: ng/mL
* ND: not detected

TABLE 4

| | Niclosamide PO 20 mg/kg | | | | | |
|---|---|---|---|---|---|---|
| PK | Reference | Reference | Example | Example | Example | Reference |
| Parameters | Example 9 | Example 10 | 4 | 5 | 6 | Example 11 |
| AUC | 105.91 | 216.05 | 723.27 | 220.08 | 421.08 | 57.31 |
| (last) | | | | | | |
| Cmax | 150.6 | 107.43 | 1030.27 | 298.21 | 848 | 37.55 |
| Tmax | 0.38 | 0.63 | 0.25 | 0.25 | 0.25 | 0.5 |
| t½ | 1.17 | 1.22 | 2.83 | 0.74 | 0.99 | 1.47 |

TABLE 5

| | |
|---|---|
| | Niclosamide PO |
| Time | 20 mg/kg, Beagle |

| h | Example 7 | Example 8 | Example 9 | Example 10 |
|---|---|---|---|---|
| 0 | ND | ND | ND | ND |
| 0.25 | 203.524 | 415.857 | 229.017 | 32.162 |
| 0.5 | 86.395 | 703.003 | 237.359 | 124.037 |
| 1 | 13.788 | 89.667 | 71.688 | 126.065 |
| 2 | 73.514 | 8.616 | 2.197 | 10.911 |
| 4 | 0.966 | 1.18 | 1.106 | 0.972 |
| 6 | 0.598 | 1.21 | 0.389 | 0.647 |
| 8 | 0.365 | 0.468 | 0.875 | 0.441 |

* Plasma concentration: ng/mL
* ND: not detected

TABLE 6

| | Niclosamide PO | | | |
|---|---|---|---|---|
| PK | 20 mg/kg | | | |
| Parameters | Example 7 | Example 8 | Example 9 | Example 10 |
| AUC (last) | 207.38 | 453.01 | 207.19 | 169.15 |
| AUC (inf) | 208.88 | 454.09 | 208.4 | 171.38 |
| Cmax | 203.52 | 703 | 237.36 | 126.07 |
| Tmax | 0.25 | 0.5 | 0.5 | 1 |
| t½ | 2.85 | 1.59 | 0.96 | 3.51 |

* AUC: ng · h/mL, Cmax: ng/mL, Tmax & t½: h

### Experimental Example 3: In Vivo Pharmacokinetic Analysis of the Compositions of the Examples, Reference Examples, and Comparative Examples (Mini Pigs)

The in vivo pharmacokinetic analysis was performed by administering the compositions of Examples 11 and 12 as a single oral dose to mini pigs, and plasma drug concentration information was obtained.

In addition, the compositions of Examples 11 and 12 were each administered at a dosage of 500 mg per head once daily.

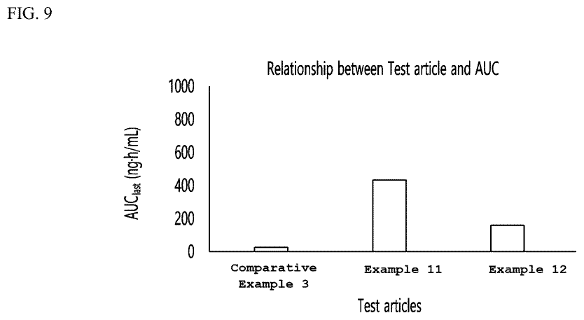

The administration results of Examples 11 and 12 are shown in FIGS. 8 and 9. The graph in FIG. 8 shows the plasma concentration of NIC over time in mini pigs. The experiment confirmed that enteric coating agents (HP-50, L-100) contribute to the bioavailability enhancement of niclosamide, and it was particularly found that the addition of the basic amino acid arginine can further maximize bioavailability. The specific pharmacokinetic parameters are listed in Tables 7 and 8. The relationship between the tested compositions and AUC is shown in FIG. 9.

TABLE 7

| Time | Niclosamide PO 500 mg/head, Mini pig | | |
|---|---|---|---|
| h | Yomesan | Example 11 | Example 12 |
| 0 | ND | ND | ND |
| 0.25 | BQL | 34.4 | 4.428 |
| 0.5 | BQL | 42.413 | 6.367 |
| 1 | BQL | 46.725 | 32.492 |
| 2 | BQL | 147.588 | 29.887 |
| 4 | 0.883 | 61.124 | 20.048 |
| 6 | 1.941 | 8.981 | 9.278 |
| 8 | 4.119 | 6.469 | 7.17 |
| 12 | 2.6 | 10.913 | 8.189 |

* Data are represented as the mean concentration (n = 2)
* Plasma concentration: ng/mL
* ND: not detected, BQL: below quantitative limit (<0.5 ng/mL)

TABLE 8

| PK | Niclosamide PO 500 mg, Mini pig | | |
|---|---|---|---|
| Parameters | yomesan | Example 11 | Example 12 |
| AUC(last) | 19.06 | 462.37 | 169.23 |
| Cmax | 4.7 | 147.59 | 32.49 |
| Tmax | 7 | 2 | 1 |
| t½ | NC | 2.51 | 4.69 |

* AUC: ng · h/mL, Cmax: ng/mL, Tmax & t½: h

### Experimental Example 4: Drug Dissolution Test

A dissolution test was conducted on Reference Examples 3 and 11, as well as Examples 5 and 6. The specific conditions for the dissolution test were as follows:

pH 1.2

After adding 2.0 g of sodium chloride to triple-distilled water, hydrochloric acid was added to adjust the pH to 1.2, and the solution was mixed to a final volume of 1 L. Then, 10 mL of Tween 60 was added and sonicated to make the solution clear. This solution (900 mL) was used in the dissolution test.

pH 6.8

After adding 6.803 g of potassium phosphate (monobasic) and 0.944 g of sodium hydroxide to triple-distilled water, the solution was mixed to a final volume of 1 L. Then, 10 mL of Tween 60 was added and sonicated to make the solution clear. This solution (900 mL) was used in the dissolution test.

### NIC Concentration

The sample was weighed to contain approximately 50 mg of NIC and used in the dissolution test.
Dissolution Apparatus Conditions
Temperature: Approximately 37.0° C.
Rotation Speed: 100 rpm
Dissolution Time: 2 hours
Sampling Times: 5 min, 10 min, 20 min, 30 min, 60 min, 90 min, 120 min

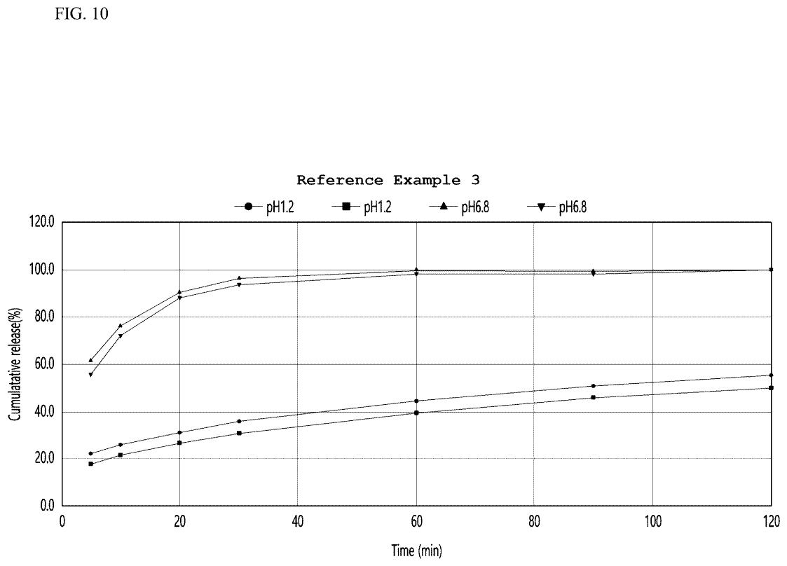

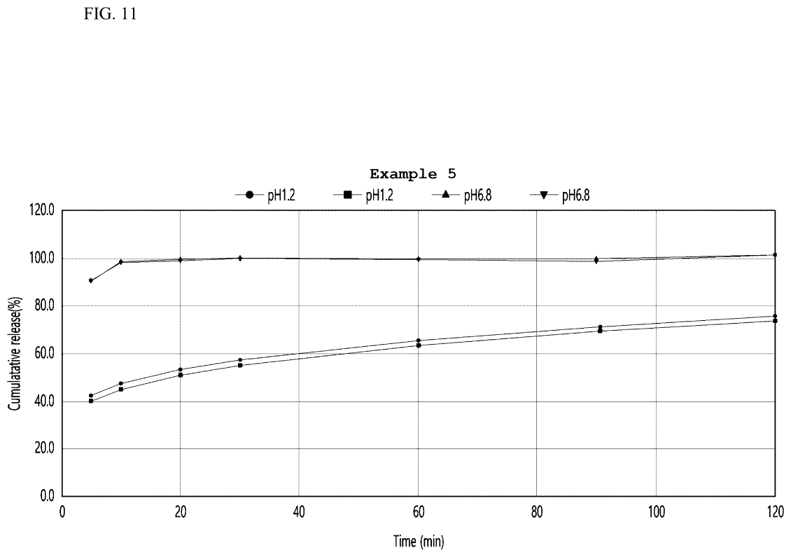

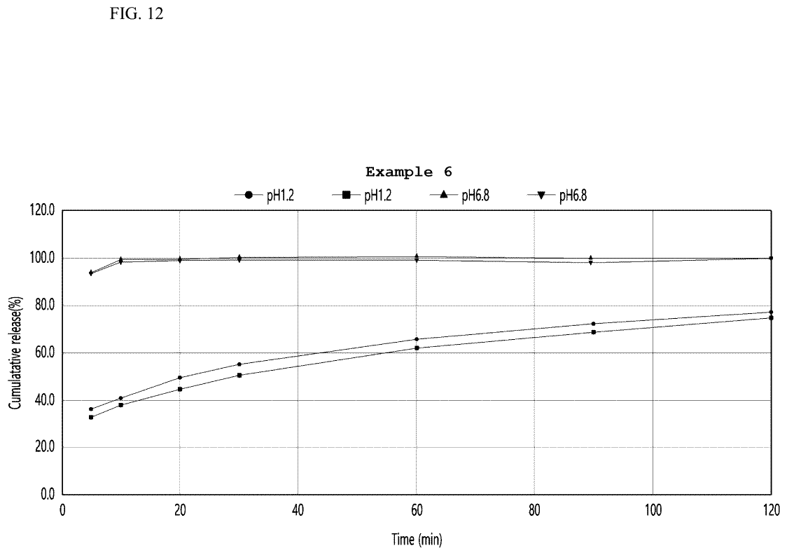

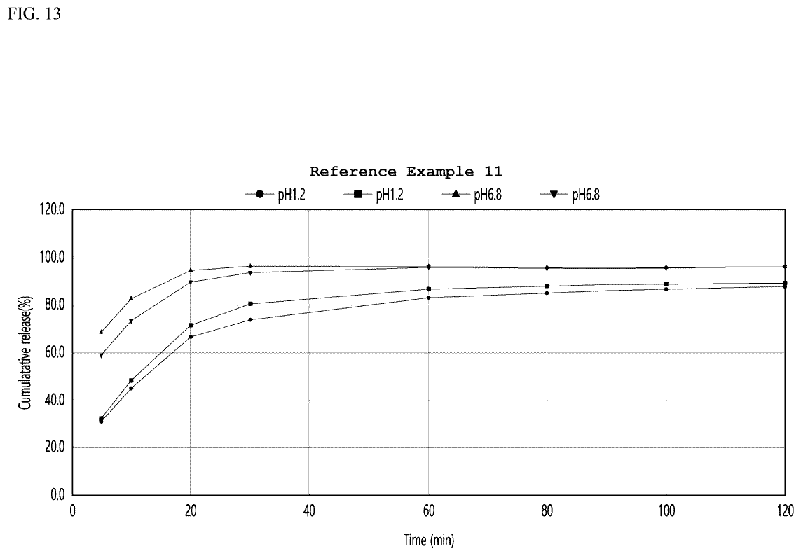

The results of the above experiment are documented in FIGS. 10 to 13. In the case of Yomesan, a commercially available niclosamide, the dissolution trends show that approximately 25% dissolution occurs in artificial gastric fluid over 2 hours, and about 18% dissolution in artificial intestinal fluid. Our invention shows higher dissolution, which plays a crucial role in increasing drug absorption.

### Experimental Example 5: Antiviral Efficacy Test of Niclosamide

An analysis was conducted to determine the antiviral effects of niclosamide and remdesivir against the Beta and Omicron variants of SARS-CoV-2. Specifically, the cytotoxicity and viral inhibition efficacy (CC50, IC50 measurements) of the compounds were analyzed.
Cells: Vero E6 (passage 35)
Virus: SARS-CoV-2
S (BetaCoV/Korea/KCDC03/20), passage 5
Omicron (B11529), passage 5
Viral Infection: MOI 0.01, 48 h
Drug Treatment Concentration: 0.097-40 μM
Viral Proliferation Analysis: qRT-PCR (Cogenbio Diagnostic Kit, E gene)
Cell Viability: WST assay

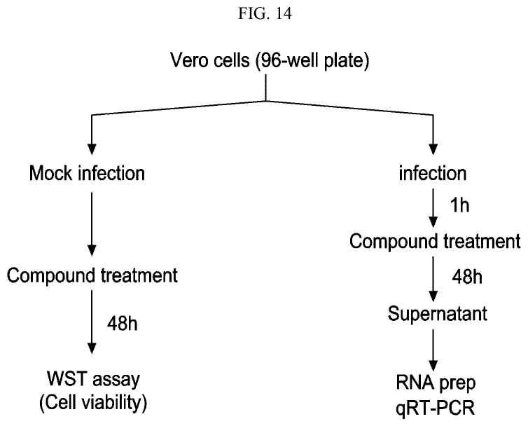

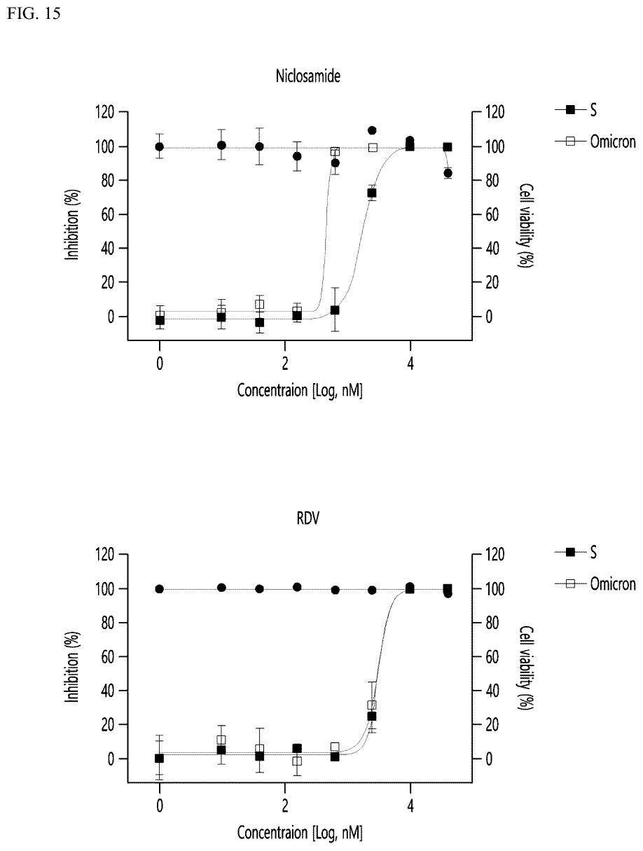

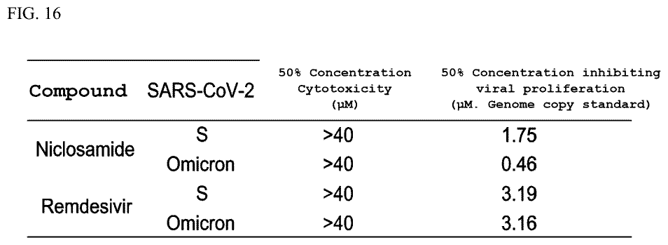

The experimental procedure is illustrated in the schematic diagram in FIG. 14. The results of analyzing the intracellular antiviral effects using niclosamide and remdesivir are shown in FIG. 15. The analysis results were based on comparing the viral genome copy to evaluate the antiviral efficacy of each compound against SARS-CoV-2, as documented in FIGS. 15 and 16. Upon reviewing FIGS. 15 and 16, it was confirmed that remdesivir, a conventional therapeutic, shows the same antiviral effect against both the Beta and Omicron variants, indicating no difference in efficacy between these variants and the original virus. In contrast, niclosamide was found to be over 1.7 times more effective in inhibiting viral proliferation compared to remdesivir. Furthermore, it was confirmed that niclosamide itself has more than four times higher antiviral efficacy against the Omicron variant compared to the Beta variant.

1. A composition for antiviral or anticancer comprising: niclosamide or a pharmaceutically acceptable salt thereof; a polyvinyl pyrrolidone-based compounds; and one or more compounds selected from among, a cellulose-based compound, a poloxamer-based compound, a polyethylene glycol-based compound, chitosan, poly-gamma-glutamic acid and xanthan gum.

2. The composition of claim 1, wherein the polyvinyl pyrrolidone-based compounds is one or more selected from the group consisting of polyvinylpyrrolidone K10, polyvinylpyrrolidone K12, polyvinylpyrrolidone K15, polyvinylpyrrolidone K17, polyvinylpyrrolidone K25, polyvinylpyrrolidone K30, polyvinylpyrrolidone K60, and polyvinylpyrrolidone K90.

3. The composition of claim 1, wherein the cellulose-based compound is one or more selected from the group consisting of hydroxypropyl methylcellulose (HPMC), hydroxyethyl cellulose, hydroxypropyl cellulose (HPC), carboxymethylcellulose (CMC), ethylcellulose (EC) and cellulose acetate (CA).

4. The composition of claim 1, wherein the polyethylene glycol-based compound is one or more selected from the group consisting of polyethylene glycol 200, polyethylene glycol 300, polyethylene glycol 400, polyethylene glycol 500, polyethylene glycol 1000, polyethylene glycol 1400, polyethylene glycol 1500, polyethylene glycol 4000, polyethylene glycol 8000, polyethylene glycol 10000, and methoxy polyethylene glycol 550.

5. The composition of claim 1, wherein the poloxamer-based compound is one or more selected from the group consisting of poloxamer 101, poloxamer 105, poloxamer 105 benzoate, poloxamer 108, poloxamer 122, poloxamer 123, poloxamer 124, poloxamer 181, poloxamer 182, poloxamer 182 dibenzoate, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407.

6. The composition of claim 1, which is one or more selected from among: a 1st combination comprising niclosamide or a pharmaceutically acceptable salt thereof, a polyvinyl pyrrolidone-based compounds and a cellulose-based compound; a 2nd combination comprising niclosamide or a pharmaceutically acceptable salt thereof, a polyvinyl pyrrolidone-based compounds and a poloxamer-based compound; a 3rd combination comprising niclosamide or a pharmaceutically acceptable salt thereof, a polyvinyl pyrrolidone-based compounds and a polyethylene glycol-based compound; a 4th combination comprising niclosamide or a pharmaceutically acceptable salt thereof, a polyvinyl pyrrolidone-based compounds and chitosan; a 5th combination comprising niclosamide or a pharmaceutically acceptable salt thereof, a polyvinyl pyrrolidone-based compounds and xanthan gum; a 6th combination comprising niclosamide or a pharmaceutically acceptable salt thereof, a polyvinyl pyrrolidone-based compounds, a cellulose-based compound and a poloxamer-based compound; a 7th combination comprising niclosamide or a pharmaceutically acceptable salt thereof, a polyvinyl pyrrolidone-based compounds, a cellulose-based compound and a polyethylene glycol-based compound; an 8th combination comprising niclosamide or a pharmaceutically acceptable salt thereof, a polyvinyl pyrrolidone-based compounds, a cellulose-based compound and chitosan; a 9th combination comprising niclosamide or a pharmaceutically acceptable salt thereof, a polyvinyl pyrrolidone-based compounds, a cellulose-based compound and xanthan gum.

7. The composition of claim 1, further comprising amino acid-based compound.

8. The composition of claim 1, further comprising amino acid-based compound.

9. The composition of claim 1, further comprising one or more of magnesium oxide (MgO), hydrotalcite, and magnesium hydroxide.

10. The composition of claim 1, further comprising one or more compounds selected from among a crystallization inhibitor, a swellable polymer, a foam generator, and a swellable excipient.

11. The pharmaceutical of claim 1, wherein the niclosamide or a pharmaceutically acceptable salt thereof and the polyvinyl pyrrolidone-based compounds satisfy the weight ratio range of 1:2 to 1:10.

12. A method for preparing a composition for antiviral or anticancer, the method comprising the steps of: preparing a first dissolved product by dissolving a polyvinyl pyrrolidone-based compounds in an organic solvent;
preparing a second dissolved product by adding and stirring niclosamide or a pharmaceutically acceptable salt thereof to the first dissolved product;
preparing a third dissolved product by further mixing one or more selected from the group consisting of a cellulose-based compound, a poloxamer-based compound, a polyethylene glycol-based compound, chitosan and xanthan gum to the second dissolved product; and
forming powders by evaporating the solvent from the third dissolved product.

13. The method for preparing a composition of claim 12, further comprising a step for mixing one or more selected from among magnesium oxide (MgO), hydrotalcite, and magnesium hydroxide to the second dissolved product.

14. The method for preparing a composition of claim 12, wherein the weight ratio range of the niclosamide or a pharmaceutically acceptable salt thereof and the polyvinyl pyrrolidone-based compounds is from 1:2 to 1:10.

15. A composition for antiviral or anticancer comprising: niclosamide or a pharmaceutically acceptable salt thereof; and one or more selected from polygamma gluthamic acid and amino acid-based compound.

16. The composition of claim 15, wherein the amino acid-based compound is one or more selected from the group consisting of glycine, alanine, valine, leucine, isoleucine, methionine, phenylalanine, tryptophan, proline, serine, threonine, cysteine, tyrosine, asparagine, glutamine, aspartic acid, glutamic acid, lysine, arginine and histidine.

17. The composition of claim 15, further comprising one or more of magnesium oxide (MgO), hydrotalcite, and magnesium hydroxide.

18. The composition of claim 15, further comprising one or more compounds selected from among a polyvinyl pyrrolidone-based compounds; a cellulose-based compound, a poloxamer-based compound, a polyethylene glycol-based compound, chitosan, poly-gamma-glutamic acid, xanthan gum, a crystallization inhibitor, a swellable polymer, a foam generator, and a swellable excipient.

19. A method of providing an antiviral or anticancer effect, the method comprising administering to a subject in need thereof the composition of claim 1.
